(19) United States
(12) Patent Application Publication
(10) Pub. No.: US 2020/0261332 A1
(43) Pub. Date: Aug. 20, 2020

(54) STABLE ANHYDROUS TOOTHPASTE CONCENTRATE FORMULATION AND METHOD OF MAKING SAME

(71) Applicant: ONE HOME BRANDS, INC., New York, NY (US)

(72) Inventor: Syed Humza NAQVI, Walnut Creek, CA (US)

(21) Appl. No.: 16/796,440

(22) Filed: Feb. 20, 2020

Related U.S. Application Data
(60) Provisional application No. 62/808,034, filed on Feb. 20, 2019.

(51) Int. Cl. A61K 8/21; A61K 8/02; A61K 8/34; A61K 8/73; A61Q 11/02

(52) U.S. Cl. CPC A61K 8/21; A61K 8/0225; A61K 8/34; A61K 2800/92; A61Q 11/02; A61K 2800/31; A61K 2800/28; A61K 8/731

(57) ABSTRACT

The invention relates to toothpaste tablets comprising a binding agent, an abrasive agent, a whitening agent, a polishing agent, a sulfate free surfactant, natural gum, sweetener, and a flavor agent.

### CROSS REFERENCE TO RELATED APPLICATION

This application claims priority to U.S. Provisional Application No. 62/808,034, filed Feb. 20, 2019, the entire content of which is incorporated herein by reference.

### BACKGROUND

Nearly all current toothpastes on the market are in paste or gel forms and packaged in a plastic tube, bottle, or pump dispenser. The problem is the packaging. Single use plastic is everywhere and it is wreaking havoc on the environment. Only 9% of all plastic is actually recycled, and packaging generates the largest portion of municipal waste (˜30%). Packaged products are inefficient for businesses and the people who buy them.

Removing the water from our formulations removes the need for single use plastic packaging and the waste that comes with it, such as packaging waste, product waste, and the waste of resources used to ship water.

Alternative toothpaste forms have been considered in the past, but have been unsuccessful as a result of formulation challenges. For example, an effervescent toothpaste tablet was developed, but it required hermetically sealed packaging to keep out moisture that would cause the effervescence to begin before the user was ready to use the toothpaste tablets. Other toothpaste tablets lacked fluoride, a key component to strong and healthy teeth and strongly recommended by the American Dental Association (ADA).

Thus, a need exists for new stable formulations of toothpaste tablets that meet the needs of consumers and the recommendations of the ADA.

### SUMMARY OF THE INVENTION

The invention relates to a stable, anhydrous toothpaste concentrate formulation. The toothpaste formulation may be in tablet form and comprise a binding agent, an abrasive agent, a whitening agent, a polishing agent, a sulfate free surfactant, natural gum, sweetener, and a flavor agent. The tablet may comprise fluoride. The fluoride may be sodium fluoride. The tablet may contain a buffer in an amount sufficient to adjust the pH when dissolved in the mouth to from about 6.0 to about 8.0. The abrasive may be sodium bicarbonate and/or magnesium stearate. The sweetener is selected from the group consisting of stevia, sorbitol, and xylitol. The amount of sweetener may range from about 0.5% to about 75% by weight. The binding agent may range from about 20% to about 80% by weight. The binding agent may be sorbitol. The amount of surfactant may range from about 1.0% to about 10% by weight, based on the weight of the tablet. The amount of abrasive may range from about 0.1% to about 30% by weight. The abrasive/polishing agent may be selected from hydrated silica and sodium bicarbonate.

In one aspect, the invention relates to a stable toothpaste tablet for use in brushing a subject's teeth that comprises a binding agent in an amount ranging from about 20% to 95% such as from 20% to 80% by weight, a surfactant in amount ranging from about 1.0% to 10% by weight, an abrasive in an amount ranging from about 0.1% to 30% such as from about 0.1% to 10% by weight, and an artificial sweetener in an amount ranging from about 0.5% to 75% such as from about 0.5% to 10% by weight. The tablet may further include fluoride in an amount ranging from about 0.15% to 0.30% by weight. The tablet may further contain a buffer in an amount sufficient to adjust the pH when dissolved in the mouth to from about 6.0 to about 8.0.

In another aspect, the invention relates to a method of making a toothpaste tablet for use in brushing a subject's teeth.

### DETAILED DESCRIPTION OF THE INVENTION

This disclosure relates to stable anhydrous toothpaste concentrate formulations. The inventors have discovered a formulation that is both good for the environment and effective for maintaining good oral hygiene. The tablet is a unit dose tablet ranging in size from about 0.20 to 0.50 inches in diameter. The advantages of this anhydrous tablet over the traditional tubed toothpastes include chemical stability, reduced packaging, and convenience for the consumer. “Anhydrous” means that the formulation contains less than about 5%, 4%, 3%, 2% or 1% by weight of water based on the weight of the concentrate formulation.

Some of the ingredients may have multiple functions. However, when two or more ingredients defined based on their functions are included in a formulation disclosed herein, the ingredients differ from each other in terms of their chemical structure. For example sorbitol can be a binding agent and a sweetener as well, but when both binding agent and sweetener are used in the description of the formulation, they intend to refer to different ingredients in terms of the chemical structure.

Specifically, the toothpaste tablets of this disclosure may include an abrasive, a whitening agent, a polishing agent, a mild sulfate free surfactant, natural gum, sweetener, flavor, and optionally fluoride.

The amount of fluoride in the tablet may range from about 0.05% to about 0.30% by weight, based on the weight of the tablet. The amount of fluoride per tablet may be about 0.05%, about 0.10%, about 0.15%, about 0.20%, about 0.25%, about 0.30%, about 0.10% to about 0.25%, about 0.15% to about 0.20%, about 0.05% to about 0.25%, about 0.05% to about 0.20%, about 0.05% to about 0.15%, about 0.10% to about 0.30%, about 0.10% to about 0.25%, about 0.10% to about 0.20%, about 0.10% to about 0.15%, about 0.15% to about 0.30%, about 0.15% to about 0.25%, or about 0.15% to about 0.20%. The fluoride may be sodium fluoride, stannous fluoride, or sodium monofluorophate. In one embodiment, the fluoride is sodium fluoride.

The tablet may contain a buffer in an amount sufficient to adjust the pH when dissolved in the mouth to from about 6.0 to about 8.0. The pH of the dissolved tablet in the mouth may be about 6.0, about 6.5, about 7.0, about 7.5, or about 8.0.

The amount of abrasive agent in the tablet may range from about 0.1% to about 30% by weight, based on the weight of the tablet. The amount of abrasive agent is sufficient to mechanically remove debris from a tooth surface and debride plaque, it may be about 0.1%, about 0.5%, about 1.0%, about 1.5%, about 2.0%, about 2.5%, about 3.0%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, about 15.0%, about 15.5%, about 16.0%, about 16.5%, about 17.0%, about 17.5%, about 18.0%, about 18.5%, about 19.0%, about 19.5%, about 20.0%, about 20.5%, about 21.0%, about 21.5%, about 22.0%, about 22.5%, about 23.0%, about 23.5%, about 24.0%, about 24.5%, about 25.0%, about 25.5%, about 26.0%, about 26.5%, about 27.0%, about 27.5%, about 28.0%, about 28.5%, about 29.0%, about 29.5%, about 30.0%, about 0.5% to about 20.0%, about 10% to about 20%, or about 1% to about 10% by weight. The toothpaste tablet may contain one or more abrasive agents, such as dehydrated silica, hydrated silica, calcium carbonate, surface treated carbonate salts or phosphates, and mixtures thereof. The tablet may contain one or more of sodium bicarbonate or Dentsil 230 (silica).

The amount of sweetener in the tablet may range from about 0.5% to about 75% by weight, based on the weight of the tablet. The amount of sweetener is an amount sufficient to adjust the flavor of the tablet when chewed by a user and may be about 0.5%, about 1%, about 5%, about 10%, about 15%, about 20%, about 25%, about 30%, about 35%, about 40%, about 45%, about 50%, about 55%, about 60%, about 65%, about 70%, about 75%, about 1% to about 75%, about 5% to about 75%, about 10% to about 75%, about 15% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 5% to about 70%, about 15% to about 65%, about 20% to about 60%, about 25% to about 55%, about 30% to about 50%, or about 35% to about 45% by weight. The toothpaste tablet may contain one or more sweeteners selected from the group consisting of polysaccharides, artificial polyols, natural polyols coming from fruits and trees, highly refined fruits (example Lou Han Gue), purified herbs, green tea, mongloicides, white tea, stevia, carrageenan, cranberry, natural pectin, alginic acid, xanthan gum, hyaluronic acid, chondroitin sulfate, gum Arabic, gum karaya, gum tradgacanth, chitosan, starch, dextrins, Neotame, Sucralose, Acefulame K, Saccharin(s), Cyclomates, Apartame, Licorice root, Glycyrrhizin extract, Citrguar gum, guar gum, lactose, glyhrician manufactured under the brand name Magnasweet and mixtures thereof. If sorbital or other binders are used, they may also function as sweeteners. The tablet may contain one or more sweeteners, such as stevia, sorbitol, or xylitol.

The amount of binding agent in the tablet may range from about 20% to about 80% by weight, based on the weight of the tablet. The amount of binding agent is suitable to form a tablet and may be about 20%, about 25%, about 30%, about 35%, about 40%, about 45%, about 50%, about 55%, about 60%, about 65%, about 70%, about 75%, about 80%, about 60% to about 80%, about 20% to about 70%, about 20% to about 60%, about 20% to about 50%, about 20% to about 40%, or about 30% to about 40%. The binding agents may be polyols, such as mannitol, maltitol, sorbitol, isomalt, erythritol and xylitol or invert sugars. Other binders may include calcium phosphate, calcium carbonate, sodium phosphate, sodium bicarbonate, sodium carbonate, sodium phosphate, potassium phosphate, potassium carbonate, polyethylene glycol and esters thereof. The toothpaste tablet may contain one or more binding agents, such as sorbitol, xylitol, dextrose, or microcrystal cellulose (MCC). A preferred binding agent may be sorbitol.

The amount of surfactant in the tablet may range from about 1.0% to about 10% by weight, based on the weight of the tablet. The amount of surfactant is an amount sufficient to reduce surface tension and increase the rate of dissolution of the tablet in contact with saliva and assist in foaming the tablet, it may be about 1%, about 2%, about 3%, about 4%, about 5%, about 6%, about 7%, about 8%, about 9%, about 10%, about 2% to about 10%, about 3% to about 10%, about 4% to about 10%, about 5% to about 10%, about 6% to about 10%, about 7% to about 10%, or about 8% to about 10%. The toothpaste tablet may contain one or more surfactants, such as nonionic surfactants or anionic surfactants. The tablet may contain sodium lauroyl glutamate, or other mild, sulfate-free surfactants.

The toothpaste tablet may contain one or more polishing agents, such as hydrated silica or sodium bicarbonate.

The toothpaste tablet may contain one or more flavoring agents, such as peppermint oil, spearmint oil, anise oil, or wintergreen oil.

The toothpaste tablet may contain a natural gum, such as carageenen.

In one aspect, the invention relates to a stable toothpaste tablet for use in brushing a subject's teeth that comprises a binding agent in an amount ranging from about 20% to 95% such as from about 20% to 95% such as from 20% to 80% by weight, a surfactant in amount ranging from about 1.0% to 10% by weight, an abrasive in an amount ranging from about 0.1% to 30% such as from about 0.1% to 10% by weight, and an artificial sweetener in an amount ranging from about 0.5% to 75% such as from about 0.5% to 10% by weight. The tablet may further include fluoride in an amount ranging from about 0.15% to 0.30% by weight. The tablet may further contain a buffer in an amount sufficient to adjust the pH when dissolved in the mouth to from about 6.0 to about 8.0. The buffer can be selected from trisodium phospahe, sodium citrate, and pyrophosphates.

In some embodiments, the tooth paste tablet comprises sorbitol at an amount from about 20% to 94%, a sulfate free surfactant at an amount from about 1% to 5%, silica or sodium bicarbonate at an amount, from 0.1 to 30%, and xylitol or stevia at an amount from about 0 to 5% by weight.

The toothpaste tablets described herein can also be used as a mouth washing tablet by dissolving the tablets in water and rinsing mouth with the aqueous solution.

### Methods for Preparing Toothpaste Tablets

The toothpaste tablets can be prepared using any suitable method. Stable anhydrous toothpaste tablet can be prepared using direct compression or wet granulation process. For this application direct compression is most preferred. The term direct compression (or direct compaction) is used to define the process by which tablets are compressed directly from powdered mixture of ingredients into a firm compact without employing the process of granulation. Powder is blended homogeneously by using a blender (V-blender, paddle blender, drum mixing). The powder blender is then charged into the hooper of tablet press. Desire weight, compression ton, & hardness of tablet are set as the tablets get compressed and come out of the tablet press.

An an example, a pre-mixture is prepared by combining flavors in a vessel by itself. Then in a mixing blender silica, the flavor pre-mixture, surfactant, carrageenan powder, and magnesium stearate are combined (Phase 1). In a separate blender, sodium bicarbonate, sweetener, binding agent, and optionally fluoride, are combined (Phase 2). The powder mixture can be emptied into lined air tight fiber drums and then loaded into a tablet press, before direct compression into tablets. The toothpaste tablets may be prepared at a temperature below about 70° F. and a relative humidity below about 25%.

The size of the toothpaste tablets may be about ¾^{th}-⅕^{th} of an inch, about ¾^{th} inch, about ⅜^{th}, or about ⅕^{th} of an inch in diameter.

The estimated hardness of the toothpaste tablets may be about 6/8 kp.

### Formats

Although described throughout the application as tablets, the stable anhydrous toothpaste concentrate formulations may be formatted as chewable tablets, granules (i.e., tooth powder), caplet/soft gel (similar to a fish oil pill), hard shell with liquid or powder in the middle (standard pill casing), a gummy (similar to a gummy bear), or a gusher (liquid in the middle).

The tablets may range in size from about 200 mg to about 5000 mg. The tablets may be about 200 mg, about 300 mg, about 400 mg, about 500 mg, about 600 mg, about 700 mg, about 800 mg, about 900 mg, or about 1000 mg. In a preferred embodiment, the tablets are round, however other geometric shapes are contemplated.

### Methods of Using Toothpaste Tablets

In one aspect, the invention relates to a method of cleaning a subject's teeth comprising (a) chewing the toothpaste tablet described herein, and optionally (b) brushing the teeth with a toothbrush.

Each individual toothpaste tablet, when exposed to saliva and lightly chewed, will dissolve into a foaming toothpaste. Upon experiencing dissolution of the toothpaste tablet, the user may proceed with brushing as usual with either an electric or manual toothbrush to generate foaming and cleaning, and then expectorating. Individual tablets may be packaged together in suitable bulk quantities, such as 60-120 tablets per package.

The toothpaste tablets may be stored in any suitable container, such as but not limited to plastic, glass, aluminum, ceramic, or acrylic container. The container may contain a desiccant. The container may be re-usable and refilled with new tablets as needed.

### EXEMPLIFICATION

Materials used in the following Examples and their sources are listed below.

### Example 1

A fluoride toothpaste tablet was produced, using the following ingredients:

TABLE 1

| Ingredients | Amount (%) |
|---|---|
| Flavor blend (natural essential oils) | 2.201 |
| Dentisil 230 (silica) | 5.0 |
| Amisoft LS-11 (surfactant) | 3.429 |
| Carrageenan powder | 2.0 |
| Magnesium Stearate | 0.5 |
| Sodium bicarbonate granular | 10.0 |
| Stevia powder organic | 0.5 |
| Sorbitol FCC crystalline coarse powder | 73.113 |
| Xylitol USP | 3.0 |
| Sodium fluoride | 0.254 |

### Example 2

Another fluoride toothpaste tablet was produced, using the following ingredients:

| | |
|---|---|
| Dentsil 230 | 1-15% |
| Sipernat 22 (Silicone Dioxide) | 0-15% |
| Ticaloid 881m Powder | 0-1.0% |
| Magnesium Stearate Ligafood MF2V | 0.1-1.0% |
| Sodium Lauryl Sarcosinate | 1-5% |
| Stevia 90% Powder Organic | 0-5% |
| Sorbitol FCC Crystalline Coarse Powder | 20-94% |
| Xylitol USP | 0-5% |
| Sodium Monofuorophosphate (88%) | 1.11% |
| Microcrystalline Cellulose MCC101 + D | 5-20% |
| Comprecel | |

The estimated hardness of the tablets was 6/8 kP. The tablets were ⅜th inch round, standard shape. The tablets were 600 mg each. Each tablet contained 15% fluoride.

1. A toothpaste tablet, comprising a binding agent, an abrasive agent, a whitening agent, a polishing agent, a sulfate free surfactant, natural gum, sweetener, and a flavor agent.

2. The tablet of claim 1, further comprising fluoride.

3. The tablet of claim 2, wherein said fluoride is sodium fluoride.

4. The tablet of claim 1, which further contains a buffer in an amount sufficient to adjust the pH when dissolved in the mouth to from about 6.0 to about 8.0.

5. The tablet of claim 1, wherein the abrasive is sodium bicarbonate and/or magnesium stearate.

6. The tablet of claim 1, wherein the sweetener is selected from the group consisting of stevia, sorbitol, and xylitol.

7. The tablet of claim 1, wherein the amount of sweetener ranges from about 0.5% to about 75% by weight.

8. The tablet of claim 1, wherein the binding agent ranges from about 20% to about 80% by weight.

9. The tablet of claim 1, wherein the binding agent is sorbitol or cellulose.

10. The tablet of claim 1, wherein the amount of surfactant ranges from about 1.0% to about 10% by weight, based on the weight of the tablet.

11. The tablet of claim 1, wherein the amount of abrasive ranges from about 0.1% to about 30% by weight.

12. The tablet of claim 1, wherein the polishing agent is selected from hydrated silica and sodium bicarbonate.

13. A toothpaste tablet for use in brushing a subject's teeth, comprising: a binding agent in an amount ranging from about 20% to 95% such as from 20% to 80% by weight, a surfactant in amount ranging from about 1.0% to 10% by weight, an abrasive in an amount ranging from about 0.1% to 30% such as from about 0.1% to 10% by weight, and an artificial sweetener in an amount ranging from about 0.5% to 75% such as from about 0.5% to 10% by weight.

14. The tablet of claim 13, which further includes fluoride in an amount ranging from 0.05% to about 0.30% by weight, based on the weight of the tablet.

15. The tablet of claim 13, comprising sorbitol at an amount from about 20% to 94%, a sulfate free surfactant at an amount from about 1% to 5%, silica or sodium bicarbonate at an amount from 0.1 to 30%, and xylitol or stevia at an amount from about 0 to 5% by weight.

16. The tablet of claim 13, which further contains a buffer in an amount sufficient to adjust the pH when dissolved in the mouth to from about 6.0 to about 8.0.

17. A method of making a toothpaste tablet for use in brushing a subject's teeth, comprising homogeneously blending the ingredients of claim 1 to form a mixture and compressing the mixture into tablets.

18. A method of making a toothpaste tablet for use in brushing a subject's teeth, comprising homogeneously blending the ingredients of claim 13 to form a mixture and compressing the mixture into tablets

19. A method of cleaning a subject's teeth comprising (a) chewing the toothpaste tablet of claim 1 and optionally (b) brushing the teeth with a toothbrush.

20. A method of making a toothpaste tablet for use in brushing a subject's teeth, comprising homogeneously blending the ingredients of claim 13 to form a mixture and compressing the mixture into tablets.
